(19) United States
(12) Patent Application Publication
(10) Pub. No.: US 2009/0202645 A1
(43) Pub. Date: Aug. 13, 2009

(54) INTRASYNOVIAL FORMULATIONS OF STANOZOLOL

(75) Inventor: Paolo Predieri, Cavriago (IT)

Correspondence Address: YOUNG & THOMPSON, 209 Madison Street, Suite 500, ALEXANDRIA, VA, 22314, US

(73) Assignee: ACME DRUGS S.R.L., Cavriago (IT)

(21) Appl. No.: 12/028,185

(22) Filed: Feb. 8, 2008

(51) Int. Cl. A61K 9/50; A61K 31/58; A61P 19/02

(52) U.S. Cl. 424/489; 514/176; 424/497

(57) ABSTRACT

Disclosed are intrasynovial formulations comprising stanozolol and a suitable carrier.

The present invention relates to pharmaceutical compositions for human and veterinary use comprising the active ingredient stanozolol for the intrasynovial administration, particularly intra-articular injection The invention also relates to a method of treatment of disorders of the joints and synovial fluid comprising the administration of stanozolol by intrasynovial injection treatment.

### PRIOR ART

Stanozolol, a synthetic androgen derived from testosterone, has to date been considered a classic anabolic steroid, and used accordingly in human and veterinary medicine.

The fibrinolytic activity of stanozolol and its oral use in the treatment of patients suffering from rheumatoid arthritis (Quarterly Journal of Medicine 58, 225, 19-27, January 1986) and venous lipodermatosclerosis (British Medical Journal, January 7-11) have also been described.

It has also been demonstrated that stanozolol possesses marked anti-inflammatory effects and trophic factor (IGF1) synthesis stimulating effects.

However, topical applications of stanozolol or other anabolic steroids as agents able to counteract damage to the synovial membrane, to the synoviocytes and articular cartilage are not known.

The synoviocytes cover the joint surface of the synovial membrane and produce synovial fluid, a substance which, by means of multiple actions, plays an essential role in the functionality and integrity of the joints.

Synovial fluid ensures the homeostasis of the joints as it lubricates the joint surfaces during movement and absorbs the mechanical stresses discharged onto the joint cartilage during walking and running, distributing them over large surfaces and thus protecting the joint tissues.

Synovial fluid ensures the functionality of the joints and protects their tissues if its viscosity and elasticity values are normal, ie. if the fluid is highly viscoelastic.

When the viscoelasticity values of the synovial fluid are reduced, as normally occurs in the course of inflammatory/degenerative joint disease, its lubricant and protective properties decline. Under these conditions, the friction and mechanical pressures exerted on the joint surfaces during movement cause inflammation, pain and premature wear, and degeneration of the joint tissues thus becomes inevitable and progressively more serious.

The synovial fluid also possesses the ability to regulate the entry of leucocytes into the joint, acting as a filter; when it loses its viscosity, the leucocytes normally restricted to the blood can invade the joint, releasing tissue-damaging enzymes which are mainly responsible for degeneration of the joint tissues, especially cartilage.

Synovial fluid is also the only source of nourishment of the chondrocytes, as the joint cartilage does not have its own blood supply and is therefore nourished directly by the blood vessels.

Normal metabolism and trophism of the synoviocytes influence the properties of the synovial fluid and regular nourishment of the chondrocytes, which is essential to the trophism, integrity and functionality of the joint cartilage.

Physical exercise also contributes to the nourishment of the chondrocytes, and consequently to the integrity of the joint cartilage; the forces that act on the cartilage during the loading and unloading stages cause compression and expansion of the cartilage, which expels and absorbs synovial fluid like a sponge. In this way the synovial fluid can penetrate into the cartilage tissue and nourish the chondrocytes; it can also remove the toxic substances produced by cell catabolism from the cartilage.

In joint disease, which can be triggered by trauma or motor hypoactivity due to a complex series of biochemical phenomena (first inflammatory and later degenerative), there is a gradual reduction in the metabolism of the synoviocytes and chondrocytes and the onset of dysmetabolic conditions; under these circumstances the tissues are unable to react by initiating the normal defensive or repair processes, so catabolic processes prevail over anabolic processes in the joint tissues.

The tissue degeneration may then progress, culminating in irreversible alteration of the synovial membrane and complete loss of joint cartilage.

EP 1502593 discloses pharmaceutical compositions comprising stanozolol for the treatment of inflammatory conditions, particularly of arthritis, osteoarthritis or canine tracheal collapse.

Ellis et al., in Agents Actions, 1992, 35, 232-237 discloses that stanozolol induces the production of PGE2 and collagenase in healthy skin fibroblast whereas no effect was found on synovial fibroblasts from rheumatoid synovial tissue.

Stepan J. et al., Medizinische Klinik, 62(38) 1967, 1470-1474, disclose that stanozolol has been used for the treatment of degenerative inflammatory disorders of muscolo-skeletal apparatus in humans specifically stating that said disorders (polyarthritis) exclusivily affect bone tissue as well as muscular tissue, namely connected with osteoporosis, osteomalacia and alteration of muscular tissues.

These conditions have nothing to do with the degeneration of articular cartilage, synovial capsule and synovial liquor which are on the contrary successfully treated by intra-synovial administration of stanozolol by means of suitable formulations, as hereinafter disclosed.

### DESCRIPTION OF THE INVENTION

It has now been found that stanozolol, unlike the majority of NSAIDs (non-steroidal anti-inflammatory drugs) and anti-inflammatory corticosteroids, does not cause the degeneration of tissue, especially cartilage tissue, but regenerates it. This is even more surprising since a skilled person would have expected that stanozolol, as an anabolic steroid, would have caused tissue damage when applied topically as a consequence of pro-apoptotic effects as reported, for instance, in J.Cell Physiol. 2001, 187(1):90:5 and Kidney Int. 2004; 65(4)1252-61.

It has also been found that stanozolol, when suitably formulated for the intrasynovial administration (intra-articular formulations), is particularly effective in treating a variety of joint disorders, and that said administration route is highly effective, especially in humans and horses.

The particular combination of anti-inflammatory effects and trophic factor synthesis stimulating effects typical of stanozolol can be advantageously exploited to prevent and treat degeneration of the joint tissues, especially the cartilage and synovial membrane, which is often present in human and animal joint diseases.

The intrasynovial compositions (intra-articular formulations), according to the invention act directly and effectively on the synoviocytes (the cells of the synovial membrane) and the chondrocytes (cartilage cells), which are closely involved in the onset and development of inflammatory/degenerative disease, as discussed above.

In this respect, in order to protect the joint tissues and promote their repair, it is essential to reduce the inflammatory processes and activate the cell metabolism, especially that of the synoviocytes and chondrocytes, thus promoting anabolic rather than catabolic processes.

When administered by the intrasynovial route (intra-articular injection), stanozolol stimulates the chondrocytes and synoviocytes without causing the adverse systemic effects typical of oral and conventional parenteral administration (e.g. intramuscular injection), even in long-term clinical use. Intrasynovial administration is particularly advantageous in patients suffering from joint disease located in a single joint or a limited number of joints.

In vitro studies of horse chondrocyte cultures have demonstrated the 5 surprising efficacy of the pharmaceutical preparations according to the invention.

Their in vitro activity has also been confirmed in laboratory animals in which joint disease was induced by surgical techniques (meniscectomy), and subsequently in horses suffering from spontaneous joint disease.

The preparations according to the invention possess a high level of local and systemic tolerability, good bioavailability for the joint tissue cells, and excellent clinical efficacy. They also guarantee that the active ingredient will remain in the treated joint for long enough to be compatible with the clinical use of the drug, without requiring excessively frequent administration.

The preparations according to the invention can be formulated as suspensions in an aqueous carrier, suspensions or solutions in an oily or alcoholic carrier, in glycosaminoglycans, especially hyaluronic acid (sodium hyaluronan), or in dimethyl sulphoxide.

Formulating stanozolol in an aqueous carrier in formulations suitable for the intrasynovial administration presents marked technical difficulties, because stanozolol is a crystalline substance insoluble in water, which means that aqueous solutions cannot be prepared. The formulations according to the invention of stanozolol in an aqueous carrier are therefore aqueous suspensions, and it is necessary to prevent the suspended crystals of the drug from causing mechanical lesions such as abrasions and erosions despite the pressures and friction generated by the movement of the joint on the surfaces of the cartilage and synovial membrane.

This problem has been solved by using micronised stanozolol crystals of a size not exceeding 100 μm, with a size distribution curve having a prevalent population of particles with a diameter of less than 20 μm and an average volume diameter D (v 0.5) equal to or less than 7.87 μm.

This particle-size distribution does not cause mechanical lesions of the 5 joint tissues, but gives the active ingredient in aqueous suspension the ability to disperse evenly in the synovial fluid and come into close contact with the chondrocytes immersed in the cartilage matrix.

The intrasynovial formulations according to the invention are also preferably characterised by moderate hygroscopicity, to prevent an increase in intra-articular fluids, distension and ectasia of the synovial capsule, with consequent stimulation of its nerve endings and acute pain. This problem can also be solved by using stanozolol having the above-mentioned particle-size characteristics and a concentration of under 50 mg to 1 ml of injectable suspension, preferably under 10 mg/ml. The lower concentration limit depends on the required dose, but can be 0.00001 mg/ml or even lower.

The concentration and consequently the dose will obviously depend on the weight and species of the patient to be treated and the type and severity of the joint disease.

In the veterinary field, the invention is particularly suitable for horses, especially racehorses, in which joint problems can be particularly critical.

A single intrasynovial injection of said formulations allows the stanozolol to be brought up to and maintained at pharmacologically active concentrations in the joint for 6-7 days, a period which is long enough to produce evident clinical benefits and fully compatible with long-term treatments.

A single administration may be sufficient in the case of acute joint disease, while cycles of 4 or more injections repeated every 7 days are required in the case of sub-acute or chronic disease; even if repeated injections are required, the 7-day interval between one injection and the next makes the treatment acceptable in terms of the patient's compliance and the risk profile associated with injections into the joint (joint contamination, septic arthritis and inflammation caused by joint punctures).

An example of the formulation of a typical preparation containing stanozolol administrable by the intrasynovial route to horses and humans, which remains in the synovial fluid for 5-7 days, is set out below:

Active ingredient: micronised stanozolol (having a prevalent population of particles with a diameter of less than 20 μm) 5 mg;

Water for injectables q.s. to 1 ml.

In the case of chronic joint disease, in view of the need for repeated injections, the availability of “retard” or slow release formulations which can be administered once only or at 3-4 week interval may be useful. Examples of such formulations include lipophilic gels with thixotropic properties, suspensions of crystals in thermoreversible gels (which are liquids at refrigerated temperature and gel at body temperature), microcapsules of microparticulate systems of the active ingredient in lipophilic coating structures based on glycerides, waxes and solid esters, and microcapsules of microparticulate systems of the active ingredient in coating structures consisting of lactic acid polymers (polylactides-PLA) and lactic and glycolic acid copolymers (polylactide-coglycolides-PLGA).

The compositions according to the invention must not contain preservatives, which generally inhibit the cell metabolism and interfere with the stimulating action that the drug is required to exert on the chondrocytes and synoviocytes.

The compositions according to the invention can be advantageously used to treat a number of pathological conditions affecting the joint.

The definition “pathological condition affecting the joint” refers to pathological conditions affecting intra-articular tissues surrounding the articular cavity (cartilages and synovial membranes) which, being in direct contact with the synovial liquor, may be stimulated by the intra-synovial route, bone and muscular tissues being excluded from said definition.

Examples of said conditions include:
Synovial fluid with poor viscosity and elasticity characteristics;
Excessively liquid synovial fluid;
Synovial fluid with low intra-articular tissue lubricating power;
Synovial fluid with low chondrocyte nourishing power;
Joints characterised by low production of synovial fluid;
Hypotrophy of the synovial membrane;
Hypofunctionality of the synoviocytes, synovial membrane and synovial capsule;
Hypofunctionality of the chondrocytes;
Low ability to repair lesions of the joints in general and the joint cartilage in particular;
Hypotrophy of the joint cartilage;
Protection of joint cartilage and joint tissue against physical, biochemical and endotoxic damage;
Dysmetabolic conditions affecting the joint tissues;
Restoration of the physiological anabolism/catabolism ratio in joint tissues characterised by the prevalence of catabolic over anabolic processes;
Restoration of the physiological environment in joints affected by inflammatory/degenerative processes.

The formulations according to the invention are more advantageous than conventional systemic (oral or intramuscular) administration of stanozolol because they guarantee a higher concentration at the site of action, greater efficacy, no undesirable side effects, and the possibility of long-term treatments without systemic effects.

1. Formulations for intra-synovial (intra-articular) use containing micronised stanozolol and a suitable carrier.

2. Formulations as claimed in claim 1, wherein stanozolol is in suspension in an aqueous carrier.

3. Formulations as claimed in claim 1, wherein stanozolol is in an oily carrier.

4. Formulations as claimed in claim 1, wherein stanozolol is in an alcoholic carrier.

5. Formulations as claimed in claim 1, wherein stanozolol is in a carrier consisting of glycosaminoglycans.

6. Formulations as claimed in claim 5 wherein the glycosaminoglycan is hyaluronic acid or sodium hyaluronate.

7. Formulations as claimed in claim 1, wherein stanozolol is in a solvent carrier.

8. Formulations as claimed in claim 7 wherein the solvent is dimethylsulfoxide.

9. Formulations as claimed in claim 1, in the form of a suspension of micronised stanozolol of dimensions not exceeding 100 μm, with a size distribution curve having a prevalent population of particles with a diameter of less than 20 μm and a mean volume diameter D (v 0.5) equal to or less than 7.87 μm.

10. Formulations as claimed in claim 1, containing up to 49 mg/ml of stanozolol.

11. Formulations as claimed in claims 1, in the form of lipophilic gels with thixotropic properties, suspensions of crystals in thermoreversible gels (which are liquids at refrigerated temperature and gel at body temperature), microcapsules of microparticulate systems of the active ingredient in lipophilic coating structures based on glycerides, waxes and solid esters, and microcapsules of microparticulate systems of the active ingredient in coating structures consisting of lactic acid polymers (polylactides-PLA) and lactic and glycolic acid copolymers (polylactide-coglycolides-PLGA).

12. A method of treatment of a subject affected by pathological conditions of the joint and synovial fluid comprising the administration of stanozolol by intrasynovial or intra-articular injection.

13. The method of claim 12 for the in situ stimulation of the chondrocytes and synoviocytes.

14. The method of claim 12 wherein the subject is a human or non-human mammal.

15. The method of claim 12, wherein the subject is an horse.

16. The method of claim 12, wherein the subject is affected by:
Synovial fluid with poor viscosity and elasticity characteristics;
Excessively liquid synovial fluid;
Synovial fluid with low intra-articular tissue lubricating power;
Synovial fluid with low chondrocyte nourishing power;
Joints characterised by low production of synovial fluid;
Hypotrophy of the synovial membrane;
Hypofunctionality of the synoviocytes, synovial membrane and synovial capsule;
Hypofunctionality of the chondrocytes;
Low ability to repair lesions of the joints in general and the joint cartilage in particular;
Hypotrophy of the joint cartilage;
Protection of joint cartilage and joint tissue against physical, biochemical and endotoxic damage;
Dysmetabolic conditions affecting the joint tissues;
Restoration of the physiological anabolism/catabolism ratio in joint tissues characterised by the prevalence of catabolic over anabolic processes;
Restoration of the physiological environment in joints affected by inflammatory/degenerative processes.
